(19) United States
(12) Patent Application Publication
(10) Pub. No.: US 2024/0350837 A1
(43) Pub. Date: Oct. 24, 2024

(54) SYSTEM AND METHOD FOR ARTIFICALLY ADJUSTING EFFECTIVE ALTITUDE

(71) Applicant: Altitude Control Technology LLC, Denver, CO (US)

(72) Inventors: William Robert Sinclair, Denver, CO (US); Kyle Benjamin Bassett, Denver, CO (US)

(21) Appl. No.: 18/640,887

(22) Filed: Apr. 19, 2024

Related U.S. Application Data
(60) Provisional application No. 63/497,374, filed on Apr. 20, 2023.

(51) Int. Cl. A62B 7/12; A47C 7/74; A47C 27/00; A47G 9/10

(52) U.S. Cl. CPC A62B 7/12; A47G 9/1045; A47C 7/744; A47C 27/007

(57) ABSTRACT

Disclosed herein is a system for artificially adjusting effective altitude, including a concentrated gas source and a concentrated gas supply line. The first end of the gas supply line is connected to the concentrated source of gas, and the second end is connected to an air diffuser. The air diffuser can be used as a cover for a pillow or other piece of furniture. The cover includes channels incorporated into the cloth that facilitate transfer of gas from an inlet to one or more diffusion sections. The diffusion sections diffuse the gas into a cloud. When a user's head is placed adjacent to the diffusion section, the user's head is enveloped in the cloud, creating an artificially adjusted altitude.

### TECHNICAL FIELD

Various implementations of the present technology relate to reducing effective altitude for high-altitude living and, in particular, to supplementing oxygen availability in natural living environments in order to enable comfortable living at high altitudes.

### BACKGROUND

Living at high altitudes for a period of time is desirable for many due to the many outdoor recreational activities available. Rock climbing, skiing, and hiking are all readily available in the mountains. While the activities available are attractive, living long- or short-term at high altitudes often affects people in negative ways. For example, people can develop altitude sickness, headaches, shortness of breath or other symptoms. At high altitudes, the available oxygen in the air is significantly reduced compared to sea-level living, at least in part due to differences in pressure. Similarly, those individuals living at or visiting lower altitudes may have a desire to adjust their effective altitude to that of a higher elevation. Several options exist for adjusting effective altitude or improving the quality of life at high elevations.

These options include medical-grade oxygen delivery systems. For example, a person can carry an oxygen tank and cannula with them through their activities to provide increased oxygen availability. Alternatively, the oxygen supply can be used only from time to time. Oxygen cannulas can produce facial discomfort, dryness, or soreness of the nasal passages and/or throat among other inconveniences. The cannulas can also be difficult to use while sleeping.

Another option is provided by companies such as Altitude Control Technology of Edwards, Colorado. Altitude Control Technology retrofits high elevation homes to incorporate supplemental oxygen supplies and various sensors to increase the oxygen content within the home. This provides oxygen comparable to a lower elevation at any time while a person is within the home. This process can be expensive and invasive and may not be feasible in all situations. A solution is desirable for a more portable solution to mimic a lower altitude environment.

### OVERVIEW

Disclosed herein is a system for artificially adjusting effective altitude, including a concentrated gas source and a concentrated gas supply line. The concentrated gas supply line includes a first end and a second end. The first end is connected to the concentrated source of gas, and the second end is connected to a diffuser. The concentrated gas supply line is configured to transfer concentrated gas from the concentrated gas source to the diffuser. The system further includes a cloth air diffuser which can be used as a cover for a pillow or other piece of furniture. The cloth cover includes channels incorporated into the cloth that facilitate transfer of gas from an inlet to one or more diffusion sections. The diffusion sections diffuse the gas into a cloud. When a user's head is placed adjacent to the diffusion section, the user's head is enveloped in the cloud, creating an artificially adjusted altitude.

### BRIEF DESCRIPTION OF THE DRAWINGS

Many aspects of the disclosure may be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views. While several embodiments are described in connection with these drawings, the disclosure is not limited to the embodiments disclosed herein. On the contrary, the intent is to cover all alternatives, modifications, and equivalents.

FIG. 1 illustrates an operational environment in an example implementation.

FIG. 2 illustrates an additional operational environment in an example implementation.

FIG. 3 illustrates an apparatus in an implementation.

FIG. 4 illustrates another apparatus in an implementation.

FIGS. 5A-5C illustrate additional apparatuses in implementations.

FIG. 6 illustrates an apparatus in an implementation.

FIG. 7 illustrates a method according to an implementation.

The drawings have not necessarily been drawn to scale. Similarly, some components and/or operations may be separated into different blocks or combined into a single block for the purposes of discussion of some of the embodiments of the present technology. Moreover, while the technology is amenable to various modifications and alternative forms, specific embodiments have been shown by way of example in the drawings and are described in detail below. The intention, however, is not to limit the technology to the particular embodiments described. On the contrary, the technology is intended to cover all modifications, equivalents, and alternatives falling within the scope of the technology as defined by the appended claims.

### DETAILED DESCRIPTION

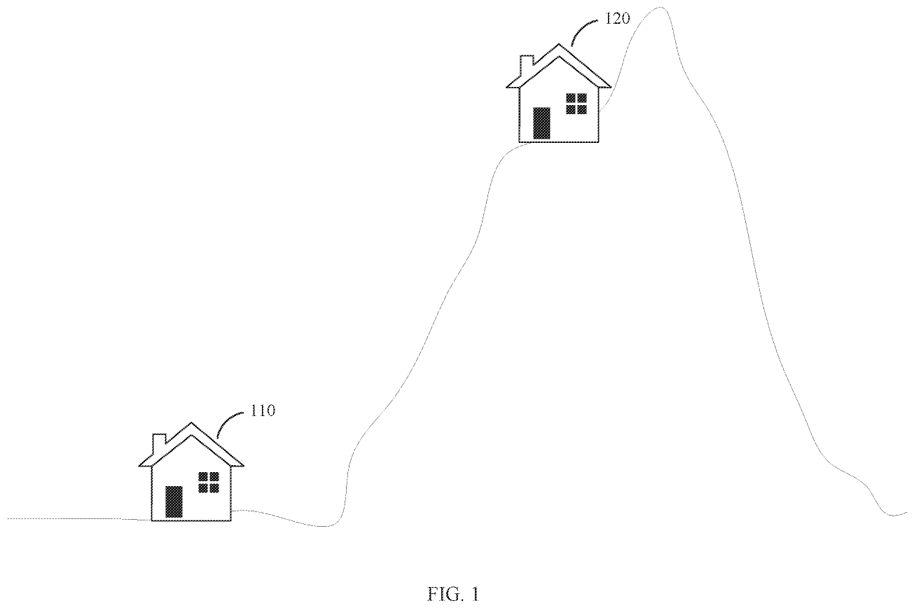

Living at high altitudes presents various problems associated with low oxygen intake. Looking at FIG. 1, house 110 is located at sea level. On average, the effective oxygen percentage available in the air at house 110 is around 21%. House 120 is located at a higher elevation. In an implementation, this could be approximately 10,000 feet above sea level. On average, the effective oxygen available in the air at house 120 is around 14%. While 10,000 feet above sea level is identified, any altitude could utilize the methods disclosed herein to improve well-being at high altitudes. At house 120, while the oxygen availability in the air is significantly reduced, most people can function relatively normally for a short time. Over time, however, the reduced oxygen may produce negative outcomes. The present methods allow for normal activity during part of the day, followed by spending time in an artificially reduced effective altitude state for a period of time. This reduced effective altitude allows the body to recuperate and can effectively promote well-being and comfort. Thus, normal activities, such as rock-climbing, skiing, hiking, etc. can be performed throughout the day, and recuperation can occur during relaxation time or sleep.

Simulated altitude adjustment may or may not include adjustment of overall air pressure. For example, house 120 may have a lower partial pressure of oxygen than house 110. Additionally, the overall air pressure at house 120 may be lower than that at house 110. In an implementation, by adjusting the partial pressure of a gas, such as oxygen or nitrogen, in the atmosphere, a different altitude can be simulated, even when the overall air pressure is not adjusted, or is minimally adjusted. Similarly, while an adjustment of the partial pressure of a gas or overall air pressure can mimic a different altitude, in an implementation, the actual adjustment may not match that of a given altitude. Rather, the adjustments may provide benefits of a lower or higher altitude without seeking to accurately replicate a particular altitude.

Similarly, many users may find a benefit to artificially increasing effective altitude. For example, a user living at house 110 may desire to encourage their body to increase oxygen holding capacity by simulating life at the altitude of house 120. Historically, for example, many athletes have chosen to live and train at high altitudes to encourage such a change. By artificially adjusting the effective altitude for a period of time each day, an athlete may remain at their low altitude home, and receive some of the benefits of high-altitude training.

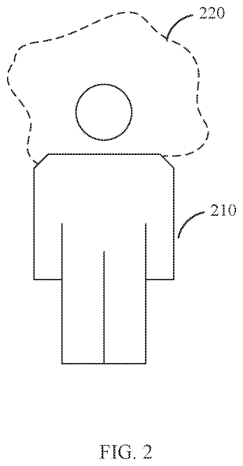

Turning to FIG. 2, an individual 210 is shown with an artificially adjusted effective altitude cloud 220. The artificially adjusted effective altitude cloud 220 can include a variety of elements adjusted to mimic adjusted altitude. By spending time in this cloud 220, the individual 210 can avoid or delay some of the negative consequences of altitude or encourage some of the positive benefits of a given altitude. The cloud 220 may, for example, include increased oxygen content. The oxygen content may be measured with various feedback sensors, as will be discussed below. In an implementation, the cloud 220 is created with a goal of mimicking a lower altitude (or possibly exceeding the oxygen content of a lower altitude), with the understanding that time spent in the cloud 220 will benefit the individual. In another implementation, the amount of oxygen provided to the individual 210 can be carefully observed and tracked to calibrate and control the amount of oxygen received by the individual 210. In another implementation, concentrated nitrogen can be provided to mimic a higher altitude environment.

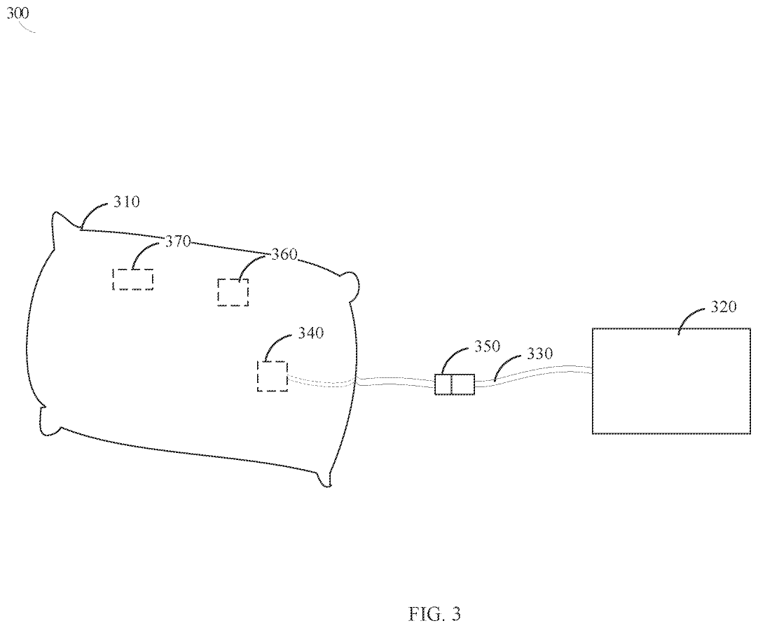

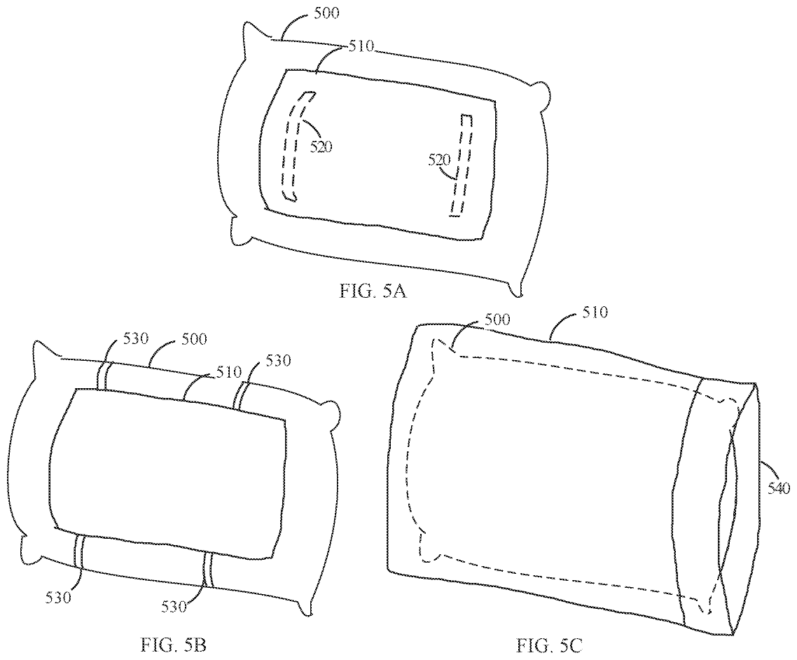

FIG. 3 illustrates a cloud apparatus 300, according to an implementation, to create the artificially adjusted effective altitude cloud 220. Pillow 310 is presented as a basis for creating the cloud 220. As most individuals 210 spend a significant amount of their day sleeping, creating a cloud 220 around the individual's head while sleeping can be an effective method to provide a recuperating adjusted affective altitude. Pillow 310 could be any type of pillow. In an implementation, pillow 310, even with the additional items discussed below, remains as similar as possible to a standard pillow, encouraging restful sleep. Pillow 310 could be a memory foam pillow, a down pillow, a synthetic foam pillow, or some other type of pillow. In an implementation, pillow 310 will conform to standard pillow sized, allowing for the use of standard sheeting, etc. In an implementation, the additions discussed below could be inserted into an existing pillow that the individual 210 may already be comfortable with. Alternatively, the additions could be provided in a cover for the pillow, as discussed further in FIGS. 5A-5C. Source 320 is a source of an air additive to make cloud 220. For example, source 320 could be a source of oxygen, nitrogen, humidity, aromatherapy, etc. In an implementation, source 320 could be an oxygen concentrator. Source 320 may be located near pillow 310 or may be located at a distance from pillow 320. For example, source 320 could be an oxygen concentrator that is located outside the room or home where pillow 310 is located. Source 320 is connected to diffuser 340 by a supply line 330. In an implementation, supply line 330 will also include an anti-tangling mechanism 350 such as a rotatable connection. This anti-tangling mechanism 350 could take many forms and allows the pillow to be moved during sleep without disturbing the production of cloud 220. Supply line 330 may be standard tubing, such as tubing used to connect oxygen tanks with breathing cannulas. Diffuser 340 can be an element with pores or micropores, either naturally or artificially created. By way of example, diffuser 340 could be a piece of limewood. In an implementation, diffuser provides resistance to the exiting gases, while dispersing the gases in a cloud around diffuser 340.

It should be noted that, while FIG. 3 only illustrates a single supply line 330 and diffuser 340, cloud apparatus 300 could include multiple supply lines 330 and/or multiple diffusers 340. For example, supply line 330 may have a fork that splits supply line 330 into two, and connects to two diffusers 340, one on each side of pillow 310. The split of the supply line 330 may be outside of pillow 310, such that a supply line 330 enters pillow 310 from each side, or the split could be within the pillow 310, such that only one supply line 330 enters into pillow 310.

Sensor 360 is also shown in pillow 310. While sensor 360 is shown attached to pillow 310, it should be understood that sensor 360 could be located separate from pillow 310, or attached to supply line 330 or diffuser 340, for example. Sensor 360 may communicate with a processor (not shown) that could be located in the system. This communication could be through wireless communication, or through a wired communication line. The wired communication line could be combined with supply line 330, for example. By way of example, sensor 360 could be an oxygen sensor, a CO2 sensor, a barometric pressure sensor, a temperature sensor (ambient air or skin), a humidity sensor, a pulse sensor, a microphone, such as to detect snoring, a sleep sensor, a heart rate sensor, and SpO2 sensor, a breath sensor, or a motion sensor, among others. While a single sensor 360 is shown, it should be understood that many sensors 360 may be included. The processor can analyze the readings from sensor 360 to adjust the cloud 220.

Stimulator 370 is also shown on pillow 370. Stimulator may also be located separate from pillow. Stimulator may be any type of external stimulation device. For example, stimulator may be a source of olfactory stimulants (lavender, chamomile, bergamot, jasmine, rose and/or sandalwood, for example), a speaker, a heating or cooling device, a structure to change sleeping position (an inflatable airbag, or motorized lever, for example) an auditory, tangible or visible alarm, a massage element or a light, for example. While a single stimulator 370 is shown, it should be understood that many stimulators 370 may be included.

While an implementation involves simply providing oxygen from source 320 through supply line 330 and diffuser 340 to create the cloud 220, other implementations involve utilizing the various sensors and stimulators to adjust the cloud 220 and/or sleep of the individual 210. In an implementation, the processor receives the readings from sensors 360, and in response to the readings from the sensors 360, adjusts the flow or temperature of gas from the source 320 and/or activates one or more of the stimulators 370.

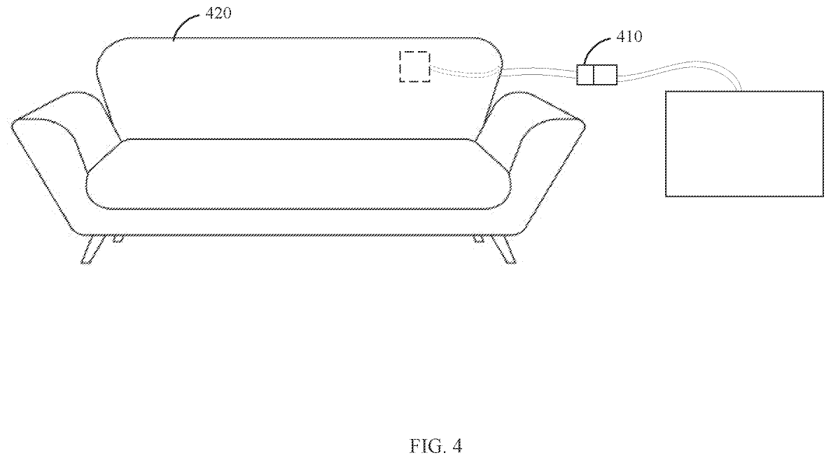

FIG. 4 shows another implementation. While individuals 210 spend much of their time sleeping, other locations may also provide good locations to create a cloud 220 for recuperation. For example, if an individual spends a lot of time sitting on the couch 420 watching TV, for example, creating an artificially reduced effective altitude cloud 220 at the couch may provide significant recuperation for the individual 210. System 410 represents the cloud apparatus 300 discussed above without the pillow 310. The system 410 could be designed and built as a system or partial system, ready to insert in a location suitable for a cloud 220. For example, system 410 may include a diffuser 340, a supply line 330, a sensor 360 and/or a stimulator 370. Supply line 330 may be created with a fitting that can attach to source 320. System 410 can then be attached, either within or on the surface of couch 420, for example. The cloud 220 can then be controlled cither manually or through a processor.

System 410 is shown in couch 420 but could be inserted into a number of other articles, such as plush furnishings. For example, System 410 could be inserted into a mattress, mattress cover, mattress pad, chair, comforter, crib, pouf, desk, or table.

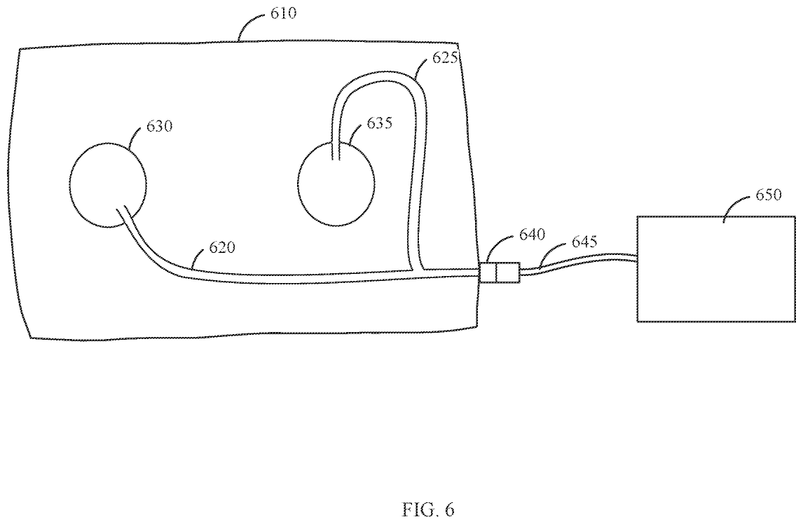

In an implementation, the apparatus is incorporated into a cover that can be removably attached to a pillow or other piece of furniture. For example, looking at FIG. 5A, furniture 500 is shown. While this is shown as a pillow, it could be another piece of furniture, such as a mattress, chair, sofa according to an implementation. For example, furniture 500 could be a piece of furniture on which a user's head will be rested. Air diffuser 510 is shown attached to furniture 500. In FIG. 5A, this removable attachment is shown by attachments 520, shown as adhesive, magnetic or hook and loop fasteners attached to air diffuser 510 and furniture 500. Air diffuser 510 can be constructed of cloth, or some other pliable material, such as plastic sheeting as will be described below with regard to FIG. 6. According to an embodiment, air diffuser could be created out of any material, hard or soft, pliable or non-pliable.

According to an implementation shown in FIG. 5B, the removable attachment of air diffuser 510 to furniture 500 can be accomplished by straps 530. Straps 530 may be designed to removably attach air diffuser 510 to furniture 500 by means of elastic, tying, buttons, snaps, hook and loop fasteners, or other such attachment mechanism.

In an implementation depicted in FIG. 5C, the removable attachment mechanism may be defined by the structure of air diffuser 510 itself. For example, back 540 is shown attached to air diffuser 510 such that air diffuser 510 and back 540 create a pocket into which furniture 500 can be inserted. This could be, for instance, a pillowcase, or a slipcover for a sofa or chair cushion.

Turning to FIG. 6, an altitude adjustment apparatus including air diffuser 610 is shown in more detail. Air diffuser 610 can be created of cloth, or some other material. The cloth, according to implementations, can be woven with a plain weave, satin weave or knit weave, or non-woven, such as felted. The cloth may be made of natural or man-made materials. Additionally, the cloth may be air (or water) permeable or non-permeable to air (or water).

In an implementation, air diffuser 610 incorporates various air channels, such as air channel 620 and air channel 625. In an implementation, air channels 620 and/or 625 are created by stitching channels in the cloth of air diffuser 610. As mentioned above, the cloth of air diffuser 610 can be a cloth that is generally impermeable to air. Thus, air introduced at one end of channel 620 or will generally proceed to the other end of the channel. In an implementation, the channel can be permeable to air or water, either due to the cloth being permeable or due to the method of attachment being permeable, such that some of the air introduced at one end of the channel will leak out during transit, but some of the air introduced into the channel will proceed to the other end of the channel. In an implementation, it may be determined that the gas lost during transit due to the permeability is beneficial, or at least not overly detrimental to the functioning of air diffuser 610.

According to implementations, channels 620 and/or 625 may be stitched, glued, fused or attached according to some other method. The cloth in the channels may be the same cloth as used elsewhere in the air diffuser, or a different cloth. By way of example, air diffuser 610 may be constructed primarily of a cloth that is highly permeable to air. Channels 620 and 625 may be constructed of a different cloth which is relatively impermeable to air. The impermeable cloth may be stitched to the permeable fabric to create an enclosed channel that is attached to air diffuser 610. In an implementation, channels 620 and 625 may be sewn onto air diffuser 610 such that at the locations of channels 620 and/or 625, there are at least three cloth layers. In another implementation, only a single layer is added to air diffuser 610 at the locations of channels 620 and/or 625 to create enclosed channels.

FIG. 6 shows that channels 620 and 625 are of roughly equal length. While this may be preferable in an implementation, it may be preferable in alternative implementations that the channels be of differing lengths or arrangements. Similarly, channels could be created with the same or differing cross-sectional area. In an implementation, by controlling the length and/or area of the channels, the designer can control the air flow through the channels.

Air that is introduced into channels 620 and 625 proceeds to diffusion areas 630 and 635. Diffusion areas 630 and 635 provide an area for the gas to diffuse into the air and create a cloud. Diffusion areas 630 and 635 can correspond to the diffuser 340 described in FIG. 3. Diffusion areas 630 and 635 can be formed from cloth that is generally highly permeable to air. Alternatively, diffusion areas 630 and 635 can be formed of cloth that is generally impermeable to air with intentional holes to allow diffusion. The holes can be created according to a design that encourages creation of an air cloud that meets design preferences. As discussed above with regard to channels 620 and 625, diffusion areas 630 and 635 may be created of the same cloth as other portions of air diffuser 610 or of a different cloth. While FIG. 6 shows 2 diffusion areas 630 and 635, it should be understood that a different number of diffusion areas may be incorporated. For example, air diffuser 610 may only have a single diffusion area 630 or may incorporate 3 or 4 or more diffusion areas. In an implementation, diffusion areas 630 and 635 are designed to create a cloud on each side of the head of a user, such that the user will breathe the enhanced air of the cloud whether the user's head is directed straight up, to the left, or to the right.

FIG. 6 further depicts an air inlet 640. Air inlet 640 may be created of a hard or soft material. In an implementation, air inlet 640 allows for easy rotation, minimizing kinking of tubing 645. Tubing 645 and air inlet 640 are attached to a source of concentrated air 650. As discussed above with regard to element 320 in FIG. 3, this could be a source of concentrated oxygen. In an implementation, this could be a concentrated source of another gas, such as nitrogen. In an implementation, several inlets may be provided and connected to the channels. In this way, multiple different gases could be connected, or a gas could be connected to a particular inlet dependent on the orientation of the air diffuser and/or a user. Air diffuser 610 may also contain a variety of air flow restrictors or valves to control air flow as needed.

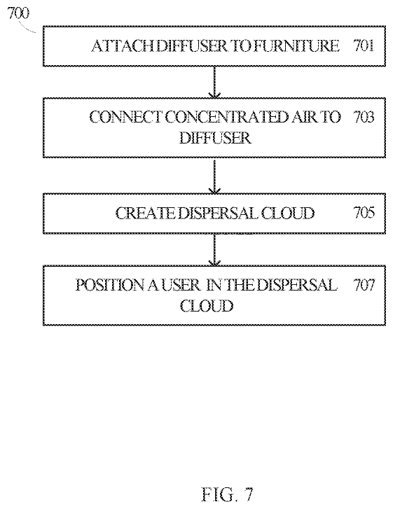

FIG. 7 depicts a method of altering a perceived altitude of a user. In step 701, an air diffuser is attached to a piece of furniture. In an implementation, the piece of furniture described is one on which a user's head is frequently laid, such as a pillow, mattress, or cushion. The air diffuser can be removably attached to the piece of furniture, or the air diffuser can be incorporated into the piece of furniture. The air diffuser can be made of one or more pieces of cloth, configured to include one or more channels and one or more diffusion areas. The channels can be formed of cloth, such as channels stitched into the cloth of the air diffuser or may be formed using tubing. The diffusion areas allow the concentrated gas to diffuse into a gaseous cloud around the diffusion areas.

In step 703, a source of concentrated air is connected to the air diffuser. In an implementation, the concentrated air may be concentrated oxygen supplied from an oxygen concentrator to create an artificially lowered equivalent altitude. In another implementation, the concentrated air may be concentrated nitrogen to create an artificially increased equivalent altitude. When the concentrated air is connected to the air diffuser, the concentrated air can pass through tubing and/or an inlet into the air diffuser.

In step 705, the concentrated air can then pass through channels in the air diffusor and diffusion areas to create one or more gaseous clouds around the air diffuser. In implementations, various sensors and monitors may be incorporated to measure the cloud produced by the air diffuser and/or adjust the amount and/or concentration of the concentrated gas supplied to the air diffuser. In this way, in an implementation, a cloud can be created that artificially mimics an altitude.

In step 707, a user is positioned in the gaseous cloud around the air diffuser. By spending time within the cloud that mimics an artificial altitude, the user can experience an artificially adjusted altitude for a period of time. In an implementation, this experience can allow the user's body to perform well at a different altitude. In various implementations, depending on the configuration of the air diffuser and the dispersal cloud, the user can be positioned in a certain position, or allowed to comfortably move between various positions within the dispersal cloud.

The included descriptions and figures depict specific embodiments to teach those skilled in the art how to make and use the best mode. For the purpose of teaching inventive principles, some conventional aspects have been simplified or omitted. Those skilled in the art will appreciate variations from these embodiments that fall within the scope of the disclosure. Those skilled in the art will also appreciate that the features described above may be combined in various ways to form multiple embodiments. As a result, the invention is not limited to the specific embodiments described above, but only by the claims and their equivalents.

What is claimed is:

1. An apparatus for altering the perceived altitude of a user, comprising:
an air inlet,
a cloth air diffuser, comprising one or more pieces of cloth, attached to comprise one or more air channels connected to the air inlet, the air channels configured to direct air from the air inlet to one or more diffusion sections;
a placement mechanism configured to removably attach the apparatus to a piece of furniture.

2. The apparatus of claim 1 wherein the air channels are defined by stitching of the one or more pieces of cloth.

3. The apparatus of claim 1 wherein the air channels are defined by gluing of the one or more pieces of cloth.

4. The apparatus of claim 1 wherein the air channels are defined by welding of the one or more pieces of cloth.

5. The apparatus of claim 1 wherein the cloth air diffuser, at least in the portions comprising the air channels, is formed of cloth that is generally impermeable to air.

6. The apparatus of claim 1 wherein the cloth air diffuser, at least in the portions comprising the air channels, is formed of cloth that is generally permeable to air.

7. The apparatus of claim 1 wherein the diffusion sections are formed from cloth that is generally permeable to air.

8. The apparatus of claim 1 wherein the diffusion sections are formed from cloth that is perforated.

9. The apparatus of claim 1 wherein the piece of furniture comprises a pillow.

10. An apparatus for altering the perceived altitude of a user, comprising:
a concentrated gas source;
a concentrated gas supply line, comprising a first end and a second end, wherein the first end is connected to the concentrated gas source, and the second end is connected to an air inlet;
a cloth air diffuser, comprising one or more pieces of cloth, attached to comprise one or more air channels connected to the air inlet, the air channels configured to direct air from the air inlet to one or more diffusion sections;
a placement mechanism configured to removably attach the apparatus to a piece of furniture.

11. The apparatus of claim 10, further comprising a controller that controls a rate of flow of concentrated gas from the concentrated gas source through the concentrated gas supply line to the air inlet.

12. The apparatus of claim 10, wherein the concentrated gas supply line further comprises a rotatable element configured to resist kinking in the concentrated gas supply line.

13. A method of altering a perceived altitude of a user, comprising:
removably attaching a cloth air diffuser to a piece of furniture;
introducing a concentrated gas into an air inlet connected to a cloth air diffuser, wherein the cloth air diffuser is configured with one or more air channels configured to direct air from the air inlet to one or more diffusion sections, creating a dispersal cloud of the concentrated gas;
positioning a user adjacent to the one or more diffusion sections and within the dispersal cloud.

14. The method of claim 13 wherein the air channels are defined by stitching of the one or more pieces of cloth.

15. The method of claim 13 wherein the air channels are defined by gluing of the one or more pieces of cloth.

16. The method of claim 13 wherein the air channels are defined by welding of the one or more pieces of cloth.

17. The method of claim 13 wherein the cloth air diffuser, at least in the portions comprising the air channels, is formed of cloth that is generally impermeable to air.

18. The method of claim 13 wherein the cloth air diffuser, at least in the portions comprising the air channels, is formed of cloth that is generally permeable to air.

19. The method of claim 13 wherein the diffusion sections are formed from cloth that is generally permeable to air.

20. The method of claim 13 wherein the diffusion sections are formed from cloth that is perforated.
